(19) United States
(12) Patent Application Publication
(10) Pub. No.: US 2018/0064730 A1
(43) Pub. Date: Mar. 8, 2018

(54) Cardiac glycosides to reduce pulmonary exacerbations and other adverse events in cystic fibrosis and other related disorders

(71) Applicant: Bette Pollard, Potomac, MD (US)

(72) Inventor: Bette Pollard, Potomac, MD (US)

(21) Appl. No.: 14/998,339

(22) Filed: Jan. 5, 2016

Related U.S. Application Data
(60) Provisional application No. 62/075,840, filed on Nov. 5, 2014.

(51) Int. Cl. A61K 31/585; A61K 31/704; C12N 15/113; A61K 45/06

(52) U.S. Cl. CPC A61K 31/585; A61K 31/704; C12Q 2600/156; A61K 45/06; C12N 15/113

(57) ABSTRACT

A small molecule inhibitor for treating a disorder in mammals, wherein the disorder is characterized by high levels of inflammatory components. In a preferred embodiment, the inhibitor comprises cardiac glycosides, also known as cardiac cardenolides. In one example, the drug digitoxin is used to treat the disorder cystic fibrosis. The compounds and methods of the invention are particularly effective for treating cystic fibrosis, a disorder or condition characterized by high levels of inflammation and IL-8.

### FIELD OF THE INVENTION

The present invention relates to immune suppression characterized by inhibiting inflammation and reducing levels of IL-8 and neutrophils, by using cardiac glycosides for treating a disorder such as cystic fibrosis (CF) and/or chronic obstructive pulmonary disorder (COPD) and other respiratory disorders, wherein the cardiac glycosides include but are not limited to digitoxin, digoxin, ouabain, oleandrin, digoxigenin, acetyldigitoxins, acetyldigoxins, cymarine, digitoxigenin, digoxigenin, medigoxin, neoconvalloside, ouabain, strophanthins, strophanthidin and acetyl-strophantidin, and other related compounds such as marinobufagenin, compositions for treatment of inflammation, and therapies that use them. These small molecules can be used alone, or they can be used in conjunction with other treatments as an adjuvant therapy. These can be used for prevention, for us as a biomarker, as a prognosticator, and for the treatment of a disease. More specifically, this invention relates to results of a clinical trial on humans having the disorder cystic fibrosis using the cardiac glycosides as the treatment for the disorder.

### BACKGROUND OF THE INVENTION

Cystic fibrosis (CF) is a fatal, autosomal, recessive genetic disease, characterized by an interleukin-8 (IL-8) related proinflammatory environment in the lung (1 Welsh) and also found throughout the body. It is the most common lethal recessive genetic disease in the United States. It occurs once in every 1500 to 2000 live births of Caucasians and once in every 17,000 live births of Afro-Americans. The majority of the patients die before the age of 40, due to pulmonary disease. The patients have early bacterial infection which starts out as Staphylococcus aureus and later becomes replaced with Pseudomonas aeruginosa. As the infections and inflammation become established in the airways, hypertrophy and hyperplasia of the mucous membranes becomes evident, ciliated cells are replaced, thick viscous airway mucosa obstructs airways and cause the inflammatory process to increase, and structural damage may occur. The primary treatment is the use of antibiotics and chest percussion to increase drainage. As the disease progresses, frequent hospitalizations are necessary. Corticosteroids and ibuprofen have been used to reduce inflammation, but they may produce adverse effects and their benefits are questionable.

The major cause of morbidity and mortality from CF is progressive pulmonary disease. This is responsible for about 95% of the mortality, and is commonly due to loss of lung function, which inexorably follows a chronic course of intrinsic inflammation, bacterial infection and airway obstruction (1 Welsh). CF is thought to be due to inactivating mutations in the chloride channel CFTR gene (2 Riordan, 3 Rommens, 4 Kerem). The protein that results from the mutant gene is called the cystic fibrosis transmembrane conductance regulator (CFTR). The CFTR gene has multiple disease causing mutations. However, the most common cystic fibrosis mutation is a deletion of 3 nucleotides that encode phenylalanine at position 508 of the CFTR amino acid sequence (ΔF508-CFTR), which is found in about 70% of cystic fibrosis cases. This mutation causes a failure of an epithelial cell chloride channel (Frizell, 1986). [ΔF508]CFTR causes defective trafficking of the mutant protein (5 Cheng, 6 Ward, 7 Ward), and also compromises chloride channel function (8 Pasyk). The CFTR mutation disease is characterized by intrinsic lung inflammation, which primarily has raised levels of IL-8. These processes are presumably causally related. Consequently, both gene therapy (9 Flotte, 10 Ruiz, 11 Walters, 12 Noone, 13 Flotte,14 Ferrari) and pharmacotherapy (15 Eidelman, 16 Eidelman, 17 Guay-Broder, 18 Jacobson, 19 Cohen, 20 Andersson, 21 McCarty, 22 Arispe, 23 Rubenstein, 24 Wang F, 25 Schultz B D, 26 Knowles M R, 27 Burns J L, 28 Geller) have been targeted towards correction of the trafficking or channel defects, and suppression of the proinflammatory phenotype of the CF airway (16, 29). Lung transplants may be used.

There is compelling clinical evidence that constitutive hypersecretion of IL-8 from epithelial cells is intrinsically responsible for the proinflammatory phenotype of the CF lung. For example, CF infants, in the absence of evidence of infection, have been reported to secrete 1000-fold elevated levels of IL8 into the airway and in the meconium (41 DiMango). In older CF patients, the new antibiotic tobramycin lowers the level of infection, while still leaving many with persistently high levels of IL-8 in the airway (29 Accurso). A particularly revealing experiment has been described by Tirouvanziam and colleagues (42 Tabary), showing that when sterile tracheal explants from fetal human CF lung are implanted into pathogen-free SCID mice, the human epithelial cells lining the implant still secrete IL8 and attract mouse leukocytes into the implant lumen. Consistently, cultured, sterile CF airway cell lines, such as IB3-1 cells, faithfully secrete equivalently massive levels of IL-8. These high levels of IL-8 secretion are substantially suppressed when the cells are repaired with un-mutated, normal, i.e. [wildtype] CFTR (16 Eidelman) These data therefore support the hypothesis that the CF lung epithelial cells secrete high levels of IL-8 without need for continuous exposure to bacteria or other pathogens.

The clinical trial “Phase II Study of Digitoxin to Treat Cystic Fibrosis;” identifier: NCT00782288 was intended to test the hypothesis that reducing the levels of inflammation such as IL-8 by treatment with cardiac glycosides, such as digitoxin, would reduce the adverse incidents that characterize cystic fibrosis, such as pulmonary exacerbations. It is generally well known that the chemical basis for the inflammatory phenotype of the CF lung is the production of massively high levels of IL-8 by CF lung epithelial cells (30 Flume, 31 Flume, 32 Flume, 33 Dean, 34 Richman-Eisenstat, 35 Francoeur, 36 Bedard, 37 Ruef, 38 DiMango, 39, Bonfield, 40 Bonfield, 41 DiMango).

IL-8 is the most powerful attractant known for inflammatory cells, such as neutrophils and macrophages, and the intrinsically high levels of IL-8 expression in the CF airway act to profoundly concentrate these inflammatory cells in the CF lung. Logically, this pathophysiological condition could be addressed either by lowering the availability of inflammatory cells for the CF lung, or by suppressing proinflammatory IL-8 production, which calls in neutrophils, by CF lung epithelium. High doses of ibuprofen have been shown to lower the systemic levels of neutrophils in CF patients, and to effectively lower inflammation in the CF lung (42 Tabary). This approach, however, faces limits due to ibuprofen toxicity. Corticosteroids have been used to reduce inflammation, but corticosteroids have profound adverse side effects which mitigate their benefits. Therefore, there is a need to have a better a drug, one that has the power to alleviate the inflammation without the profound adverse side effects.

Inflammation is the body's attempt at self-protection. It is a complex of reactions to the affected blood vessels and adjacent tissues in response to an injury or abnormal stimulation. It leads to the accumulation of fluid and blood cells at the site of injury; this response is meant to aid the injury. However, the inflammatory process can cause harm if it becomes chronic. Cystic fibrosis and other diseases result from an inflammatory component. These diseases include, but are not limited to, chronic obstructive pulmonary disease (COPD), asthma, smoke inhalation, burns, chronic bronchitis, chronic granulomatous diseases such as tuberculosis, leprosy, sarcoidosis, and silicosis, nephritis, amyloidosis, rheumatoid arthritis, ankylosing spondylitis, scleroderma, lupus, inflammatory bowel disease, cholitis, ulcers, celiac disease, Sjorgen's syndrome, Reiter's syndrome, psoriasis, pelvic inflammatory disease, orbital inflammatory disease, thromobotic disease, atopic dermatitis and contact dermatitis, Type I & II diabetes, Parkinson's disease, Alzheimer's disease, Huntington's disease, adrenoleukodystrophy, amyotrophic lateral sclerosis, retinitis pigmentosa, polylutamine disease, Bovine spongiform encephalopathy, alpha one antitrypsin deficiency, ABC A3 deficiency, short chain acyl CoA dehydrogenase deficiency, inclusion body myositis, cancer, and others.

Commonly experienced pulmonary and upper respiratory adverse exacerbations in CF. Cystic fibrosis is a complex multi-organ disease in which lung disease accounts for nearly 85% of the mortality [30]. During the course of disease, pulmonary exacerbations and adverse events associated with the deteriorating lung include: infection, airway obstruction associated with thickened secretions and cellular debris, bronchial hyperactivity, increased cough, increased sputum production, shortness of breath, chest pain, loss of appetite, loss of weight, and lung function decline [31]. Included in this list are also hemoptysis and pneumothorax [32].

IL-8 is the principal proinflammatory mediator in the CF lung. The chemical basis for the inflammatory phenotype of the CF lung is believed to be the production of massively high levels of IL-8 by CF lung epithelial cells [33-41, Di Mango, 42 Tbary, 43 Briars, 44 Tirouvanziam]. IL-8 is the most powerful attractant known for inflammatory cells such as neutrophils and macrophages, and the intrinsically high levels of IL-8 expression in the CF airway act to profoundly concentrate these inflammatory cells in the CF lung. Logically, this pathophysiological condition could be addressed either by lowering the availability of inflammatory cells for the CF lung, or by suppressing proinflammatory IL-8 production by CF lung epithelium. High doses of ibuprofen have been shown to lower the systemic levels of neurophils in CF patients, and to effectively lower inflammation in the CF lung [45]. This approach, however, faces limits due to ibuprofen toxicity.

There is compelling clinical evidence that constitutive hypersecretion of IL-8 from epithelial cells is intrinsically responsible for the proinflammatory phenotype of the CF lung. For example, CF infants, in the absence of evidence of infection, have been reported to secrete 1000-fold elevated levels of IL8 into the airway and in the meconium [43]. In older CF patients, the new antibiotic tobramycin lowers the level of infection, while still leaving many with persistently high levels of IL-8 in the airway [29]. A particularly revealing experiment has been described by Tirouvanziam and colleagues [44], showing that when sterile tracheal explants from fetal human CF lung are implanted into pathogen-free SCID mice, the human epithelial cells lining the implant still secrete IL8 and attract mouse leukocytes into the implant lumen. Consistently, cultured sterile CF airway cell lines such as IB3-1 faithfully secrete equivalently massive levels of IL-8. These high levels of IL-8 secretion are substantially suppressed when the cells are repaired with [wildtype] CFTR [16]. These data therefore support the hypothesis that the CF lung epithelial cells secrete high levels of IL-8 without need for continuous exposure to bacteria or other pathogens.

Analysis of cardiac glycoside activities identifies a structure-activity-relationship for IL-8 suppression. Applicant has previously reported that the cardiac glycoside digitoxin potently inhibits constitutive hypersecretion of IL-8 from CF lung epithelial cells [46 Srivastava, et al]. Initial pharmacodynamic studies indicated that digitoxin was the most potent of a series of tested cardiac glycosides. As indicated in this report [46], and in Table 1, the digitoxin concentration causing a 50% reduction of IL-8 secretion (K50) is ca. 0.9 nM (range: Lower. 0.8; upper. 0.9). Using all the data we were also able to derive a structure-activity relationship (see Table 1). The structures of these cardiac glycosides are shown in FIGS. 1A to 1H. Briefly, suppression of IL-8 secretion from CF lung epithelial cells is optimally promoted by the presence of glycosyl moieties at the 3-position of the cholesterol nucleus, and by the absence of oxygen-containing substitutions at or near the 12-position on the C ring. Activity declines when glycosyl moieties are absent from the 3-position, and when oxygen-containing substitutions are made at or near the 12-position on the C ring. Activity is altogether lost (viz, species VIII of reference #46) when a bulky equatorial acetyl group is substituted at the 12-position. We therefore give the equatorial 12-position and its neighbors crucial negative pharmacophoric importance, and glycosidic substitution at the 3-position positive pharmacophoric importance for the control of IL-8 secretion from CF lung epithelial cells.

TABLE 1
Pharmacodynamics of Cardiac Cardenolides

| | |
|---|---|
| K50 | Range |

| | Drug | (nM) | Lower | Upper |
|---|---|---|---|---|
| I | Oleandrin | 2.0 | 1.9 | 2.0 |
| II | Digitoxin | 0.9 | 0.8 | 0.9 |
| II | Digoxin | 27.1 | 25.2 | 27.9 |
| IV | Ouabain | 7.9 | 7.2 | 8.2 |
| V | Digoxigenin | 34.1 | 30.8 | 35.6 |
| VI | Digitoxigenin | 73.6 | 68.6 | 78.5 |
| VII | Acetyl Strophanthidin | 116.6 | 102.1 | 116.9 |
| VIII | Digitoxigenin 3,12-diAc | N/A | N/A | N/A |

Digitoxin suppresses IL-8 in CF cells by blocking the TNFα/NFκB signaling pathway. Previously published data had implicated a dysfunctional TNFα/NFκB signaling pathway as responsible for the pro-inflammatory phenotype of the CF lung. To test whether this pathway might be a target for digitoxin, a Reverse Phase Protein Microarray was prepared of IB3-1 and CFTR-repaired IB3-1/S9 cells treated with ID90 concentrations of all of the active cardiac glycosides. ID90 means a concentration of drug that blocks 90% of IL-8 secretion from the 1B3-1 cells. It was found that digitoxin and the other active species somewhat suppressed phosphorylation of IKKα, and significantly blocked CF-related constitutive phosphorylation of IKBα, and NFκB,p65. It was also found that TRADD has special significance for the CF airway inflammation because digitoxin blocks TNFalpha-activated NFκB activation and IL-8 expression in CF airway epithelial cells. (46 Srivastava et al, 2004) (47 Yang Q, et al, 2005). Based on these discoveries, digitoxin has been tested as a candidate therapy for CF in CF patients (Pollard B S, IND #70,279).

Increased expression of TRADD is associated with a greater rate of decline in CF lung function. FIG. 3A shows that in those CF patients whose TRADD mRNA levels in bronchial brush biopsies are relatively high, the rate of lung function decline is faster. Similarly, as shown in FIG. 4A, in those patients whose IL-8 mRNA levels in the same brush biopsies are relatively high, the rate of lung function decline is also faster. Trend-lines are based on data for female CF patients, who make up the majority of the patients. FIG. 5 shows that there is a high degree of correlation between mRNA levels of IL-8 and TRADD, across the entire range of FEV 1, % Predicted values (R2=0.81). These data thus show there is a close and fundamental relationship between TRADD, the first intracellular adapter protein for the TNFR (TNF Receptor), and hyper-expression of IL-8, the classical marker for CF airway inflammation. As said above, we have shown that the cardiac glycoside, digitoxin, blocks the interaction between the TNF-alpha-activated TNFR and TRADD.

The mutant CFTR protein is thought to cause hyper-responsiveness to bacteria, virus, fungi and molds in the airway which activate NFκB-dependent expression in the CF lung epithelial cells of pro-inflammatory mediators such as IL-8, IL-6, TNFalpha, IL-1Beta, and others. These pro-inflammatory proteins attract, in some instances produce, neutrophils, macrophages, and NK T cells in the airway. Too many of these becomes destructive.

The Mucosa-associated Lymphoid Tissue (MALT) is the largest mammalian lymphoid organ system and in an adult comprises 80% of all lymphoid tissue. The B cells are there for mucosal immunity, and the humoral mediators of immunity are IgA and IgM. Humans synthesize more IgA than all the other antibody classes combined because it guards the mucosal surfaces of the body. Mucins are secreted by goblet cells and mucous glands of the respiratory mucosa. Cilia, which sit on the epithelial cells, should be able to sweep the mucins out on a layer of airway surface liquid (ASL) out and upwards to the where they can be either expectorated or swallowed and taken to the acidic stomach for destruction.

In cystic fibrosis, the sustained secretion of IL-8 from the CF epithelial cells results in sustained influx of neutrophils, which should engulf any organisms it finds in the airway. In non-CF people, the chloride channel is used to synthesize hypochlorous acid (HOCl), which along with myeloperoxidase will kill the engulfed organisms. In CF people, the neutrophils use the mutant chloride channel does not work correctly. Therefore, it is only 50% effective as the normal channel would be, and the bacteria are not killed as easily as they would be in a non-CF person.

The neutrophils and the IL-8 continue to increase, and the neutrophils even synthesize more IL-8. The CF neutrophils make more neutrophil elastase from the phagosomes that is trying to eat them. The neutrophil elastase is a powerful protease which destroys epithelial cells, neutrophils and other cells, causing the release of large amounts of DNA. The DNA entangles bacteria, and mixes with mucins, and the material blocks the airway. Some of this can be removed by a drug, Pulmozyme (DNAse 1), to hydrolyze the DNA. Hypertonic saline inhalation can also elicit induced sputum production, to remove some of the mucin-DNA mass.

In CF, the airway surface liquid is reduced in amount, and it inefficient in supporting cilia-mediated movement of mucus out of the airway, and it is low in chloride. The chloride would have come from the CF epithelial cells into the airway, but it is not secreted because the chloride channel on the membrane is missing. Thus, the defensins, proteins secreted from epithelial cells to kill bacteria, which need high amounts of chloride, are not able to kill bacteria as they should. Also, the chloride in the CF airway, which should be used by neutrophils is not available.

Cardiac glycosides have been administered to CF patients in the past. With respect to the possibility of using cardiac glycosides for therapy of CF, there actually is a substantial history in the literature with digoxin (III, K50=27 nM). Peckham and colleagues [48 Peckman DG] gave what we now calculate to be “low” doses of digoxin (2-3 nM final circulatory concentration) to 11 CF and 11 control children for two weeks. Seven days after initiation of treatment, the nasal potential difference (PD) in the CF children was reduced relative to time zero or placebo control. However, based on the P value (0.06) the difference was deemed not significant. Coats and colleagues [49 Coates A L] reported that digoxin made no difference in exercise capacity or exercising cardiac function in CF vs control children. Moss and colleagues [50 Moss A J] reported that the absorption of digoxin was no different from controls when tested on CF children. Digitoxin (II, K50=0.9 nM), the most potent in term of constitutive IL-8 suppression, has not been reportedly tested on CF patients. However, from the vantage point of suppression of IL-8 secretion by CF lung cells, “high” concentrations of digitoxin, which we calculate to be ca. 30 nM in the circulation, have been tested on control patients, with only modest, or in many cases, undetectable side effects [51 Kirch W, 52 Grossmann M, 53 Grossmann M, 54 Grille W, 55 Duncker G I]. We therefore conclude that drugs in the class of cardiac glycosides appear to be safe for CF patients, and that a successful clinical trial may lead to a therapeutic role for digitoxin by suppressing the proinflammatory phenotype of the CF lung.

Summary of previous human experience with digitoxin. The earliest documented use of digitalis in humans dates to 1250 AD. Extracts of the foxglove leaf, later termed digitalis by Fuchsius, are mentioned in the writings of Welsh physicians in ca. 1250 A.D. [56 Hoffman] In 1785 William Wuthering published his “An Account of the Foxglove and Some of its Medical Uses: with Practical Remarks on Dropsy and Other Diseases”. In these earliest studies the term “digitalis” represented a mixture of what are now termed cardiac glycosides, principally including digoxin and digitoxin. The therapeutic action on the heart was until recently thought to be due to inhibition of NaKATPase. However, it is now thought that the action of cardiac glycosides on this enzyme is an aspect of drug toxicity, not efficacy for treating heart failure. The therapeutic mechanism is presently still not known [57 Kelly R A].

Clinical Pharmacology of Digitoxin.

(A) General properties of digitoxin: When given by mouth, digitoxin is completely absorbed, and is 100% bioavailable [56 Hoffman B F, 57 Kelly R A, 58 Reference P D R]. Absorption of digitoxin can be retarded by the presence of food in the GI tract, by delayed gastric emptying, and by malabsorption syndromes. Digitoxin is 90% bound to blood and tissue proteins, and it has a half-life of 7-9 days. Steady-state concentrations in the plasma are therefore attained slowly. Protein-bound digitoxin is in equilibrium with free digitoxin in the blood. Digitoxin is distributed to most parts of the body, and at equilibrium, concentrations in the heart are 15-30 fold that in the plasma. The concentration in skeletal muscle and many other organs is approximately half that of the heart. Digitoxin is metabolized in the liver, and the only active metabolite is a minor product, digoxin. That portion of digitoxin that is not metabolized is excreted in the bile to the intestines. The small amount of digoxin, as well as many of the inactive metabolites obtained by metabolic transformation of digitoxin, is rapidly excreted by the kidneys.

(B) Therapeutic uses of digitoxin: At present, the therapeutic uses of digitoxin (and digoxin) are for the treatment of heart failure [56-58]. Until recently, the only digitoxin preparation available in the U.S. was Crystadigin (Eli Lilly, reference PDF 58). Now only the short-acting digoxin is approved in the U.S. This inventor's plan to test digitoxin for treating cystic fibrosis is entirely novel.

(C) Toxicity of digitoxin in cardiac patients and normal controls: In the foregoing descriptions, a distinction is made between toxicity in cardiac patients and toxicity in normal controls. The reason is that it has been observed that the typical adverse events with digitalis mostly affect cardiac patients. Persons with normal hearts are typically resistant, and children with normal hearts are more resistant than adults. This is particularly relevant to our proposal to treat cystic fibrosis patients with digitoxin, since from a cardiac view most CF patients can be considered “normal”.

(D) Digitoxin toxicity in cardiac patients: There is little evidence that excessive amounts of digitalis in patients have direct, deleterious effects on the mechanical activity of the heart [56]. Those concentrations in blood associated with toxicity typically cause abnormalities of cardiac rhythm and disturbances of A-V conduction, including complete A-V block. The development of A-V block in patients can be due either to the vagal effects of digitalis (i.e., overcome with atropine) or to direct action on the A-V node. In patients, digitalis can cause marked sinus bradycardia and can also bring about complete S-A block. Toxicity in patients can also be manifested as disturbances of atrial rhythm, including premature depolarizations and paroxysmal and non-paroxysmal supraventricular tachycardia.

(E) Other toxic effects of digitalis in patients can include gastrointestinal effects such as anorexia, nausea and vomiting, diarrhea, abdominal discomfort and pain. Neurological effects can include headache, fatigue, malaise, and drowsiness. Mental symptoms can include disorientation, confusion, aphasia and even delirium and hallucinations, particularly in older patients. Vision can be blurred, and color vision can be disturbed, with yellow and green chromoatopsia most common. Finally, instances of gynecomastia have also been observed in patients.

The preceding description of toxicity in cardiac failure patients is for “digitalis”, without discrimination between digoxin and digitoxin. However, since applicant is proposing a study with digitoxin, it is worth commenting that a recent European study on heart failure patients found that the incidence of toxic effects from digitoxin were lower than with digoxin [59]. An equivalent study in the U.S. has recently been published in a 1995-1998 retrospective of heart failure patients, finding an approximately three-fold lower incidence of adverse events in patients taking digitoxin compared to patients taking digoxin [60].

Digitoxin toxicity in normal subjects, including those with cystic fibrosis: In fact, there is very little toxicity. An important point of about cardiac toxicity is that the likelihood and the probability of severity of arrhythmia are direct related to the severity of the underlying cardiac disease. To quote directly from Hoffman and Bigger [56], “if normal subjects with normal hearts ingest large but not lethal quantities of digitalis, either in an attempt at suicide or by accident, premature impulses and rapid arrhythmias are infrequent. In the latter case, the typical findings are sinus bradycardia and A-V block. These disturbances probably result in large part from a marked increase in the concentration of potassium in the plasma that is caused by severe acute intoxication with digitalis Infants and children seem to tolerate higher concentrations of digitalis in their plasma and myocardium than do adults”.

With respect to the possibility of using cardiac glycosides for therapy of CF, one can consider the patients to be tested as “normal subjects” from the vantage point of the state of their hearts. In retrospect, there actually is a substantial history in the literature with digoxin (III, K50=27 nM). Peckham and colleagues [48] gave what we now calculate to be “low” doses of digoxin (2-3 nM final circulatory concentration) to 11 CF and 11 control children for two weeks. Seven days after initiation of treatment, the nasal potential difference (PD) in the CF children was reduced relative to time zero or placebo control. However, based on the P value (0.06) the difference was deemed not significant. Coats and colleagues [49] reported that digoxin made no difference in exercise capacity or exercising cardiac function in CF vs control children. Moss and colleagues [50] reported that the absorption of digoxin was no different from controls when tested on CF children. Digitoxin (II, K50=0.9 nM), the most potent in term of constitutive IL-8 suppression, has not been reportedly tested on CF patients. However, from the vantage point of suppression of IL-8 secretion by CF lung cells, “high” concentrations of digitoxin, which we calculate to be ca. 30 nM in the circulation, have been tested on control patients, with only modest, or in many cases, undetectable side effects [51-55].

Drug interactions with digitoxin: With the caveat that the majority of drug interactions have been documented only in cardiac patients, the following interactions have been noted. Although not specifically seen in normal subjects, they stand as considerations to be kept in mind when testing on CF patients.

(A) Diuretics: Spironolactone has both positive and negative effects on digitalis toxicity. Diuretics which have hypokalemic and hypovolemic consequences can potentiate digitalis toxicity. These include thiazide and loop diuretics. The antibiotic amphotericin B can be a cause of hypokalemia. Other drugs whose potentiation of digitoxin activity occurs through these and related mechanisms include quinidine, verapamil, diltiazem, amiodarone, flecainide, and corticosteroids.

(B) Beta blockers: β-adrenergic agonists have the ability of inhibiting A-V nodal conduction, thereby increasing the likelihood of digitalis related arrhythmias.

(C) Non-depolarizing muscle relaxants and succinylcholine: cardiac arrhythmias can be enhanced in the simultaneous presence of digitalis.

(D) Amiloride: This compound is known to decrease the inotropic effects of digoxin. Its potential effects on digitoxin are not available in the literature. This detail is mentioned here not only for completeness but also because amiloride has been used as an experimental treatment in the past for CF.

(E) Sympathomimetics: Concomitant use with digitoxin can increase the risk of cardiac arrhythmias because both enhance ectopic pacemaker activity.

(F) Drugs than enhance hepatic microsomal enzymes: Drugs such as phenylbutazone, phenobarbital, phenytoin, and rifampin can speed the metabolism of digitoxin, thereby appearing to reduce potency. Thyroid hormone has also been reported to have this effect.

Reversal of digitoxin toxicity: The antidote for digitoxin toxicity is anti-digitoxin immunotherapy. An antibody against the pharmacophore for both digoxin and digitoxin has been prepared, and a preparation of Fab fragments is available in most poison control centers. The effectiveness and safety of anti-digoxin (and anti-digitoxin) have been fully established in both adults and children [57]. A full neutralizing dose can be administered intravenously in a saline solution over 30-60 minutes. A comprehensive review of the treatment of digitalis toxicity has been published [61].

We therefore conclude that drugs in the class of cardiac glycosides appear to be safe for CF patients, and that a successful clinical trial may lead to a therapeutic role for digitoxin by suppressing the proinflammatory phenotype of the CF lung.

### SUMMARY OF THE INVENTION

Applicant has previously reported that the cardiac glycoside digitoxin potently inhibits constitutive hypersecretion of IL-8 from CF lung epithelial cells (46). Initial pharmacodynamic studies indicated that digitoxin was the most potent of a series of tested cardiac glycosides. As indicated in this report (46), and in Table 1, the digitoxin concentration causing a 50% reduction of IL-8 secretion (K50) is ca. 0.9 nM (range: Lower, 0.8; upper, 0.9). Using all the data we were also able to derive a structure-activity relationship (see Table 1). The structures of these cardiac glycosides are shown in FIGS. 1A to 1H. Briefly, suppression of IL-8 secretion from CF lung epithelial cells is optimally promoted by the presence of glycosyl moieties at the 3-position of the cholesterol nucleus, and by the absence of oxygen-containing substitutions at or near the 12-position on the C ring. Activity declines when glycosyl moieties are absent from the 3-position, and when oxygen-containing substitutions are made at or near the 12-position on the C ring. Activity is altogether lost (viz, species VIII of reference #46) when a bulky equatorial acetyl group is substituted at the 12-position. We therefore give the equatorial 12-position, and its neighbors, crucial negative pharmacophoric importance, and glycosidic substitution at the 3-position positive pharmacophoric importance for the control of IL-8 secretion from CF lung epithelial cells.

The present invention is directed to the use of cardiac glycosides for the purpose of reducing pulmonary exacerbations in patients with cystic fibrosis, and other forms of acute or chronic obstructive pulmonary disease, which have the structures shown in Table 1.

Additionally, the present invention is directed to the use of cardiac glycosides for the purpose of reducing pulmonary inflammation and exacerbations in influenza. Just as in the experiments for cystic fibrosis, the cytokines and chemokines that are key markers of lung inflammation were significantly reduced. See FIGS. 2A to 2H.

The experimental work herein relates to treating a disorder characterized by inflammation having a component of raised chemokines/cytokines such as but not limited to IL-8, which get reduced by treatment with cardiac glycoside drugs. It was determined the source of the IL-8 cytokine/chemokine in the cystic fibrosis cells is from the NFκB pathway (Srivastava, Eidelman O. Zhang, J, Paweletz C, Caohuy H., Yang Q., Jacobson, K, Heldman E., Huang W., Jozwik C., Pollard B S., and Pollard, H B (2004) (U.S. Pat. No. 8,569,248: Inventor B S Pollard). In addition to treating cystic fibrosis, these cardiac glycoside drugs can be used to treat diseases such as but not limited to cystic fibrosis, Chronic Obstructive Pulmonary Disease (COPD), bronchiectasis (a viral lung disease), diabetes, Parkinson's disease, Alzheimer's disease, surfactant protein C deficiency, ABCA3 deficiency, Huntington's disease, adrenoleukodystrophy, bovine Spongiform encephalopathy, alpha one antitrypsin deficiency, short chain acyl CoA dehydrogenase deficiency, inclusion body myositis, chronic bronchitis, chronic sinusitis, other mucosal diseases such as but not limited to inflammatory bowel disease, ulcerative colitis, Crohn's disease, celiac disease, brain injury such as those from stroke, blast, accident, injury, smoke inhalation, cancer, atherosclerosis, rheumatoid arthritis, periodontitis, hay fever, and others.

### BRIEF DESCRIPTION OF THE DRAWINGS

The foregoing, as well as other objects and advantages of the invention, will become apparent from the following detailed description when taken in conjunction with the accompanying drawings, wherein like reference characters designate like parts throughout the several views, and wherein:

FIG. 1A depicts the structure of oleandrin.

FIG. 1B depicts the structure of digitoxin.

FIG. 1C depicts the structure of digoxin.

FIG. 1D depicts the structure of ouabain.

FIG. 1E depicts the structure of digoxigenin.

FIG. 1F depicts the structure of digitoxigenin.

FIG. 1G depicts the structure of acetyl strophanthidin.

FIG. 1H depicts the structure of digitoxigenin 3,12-diAc.

FIGS. 2A to 2H show how the cytokines and chemokines that are key markers of lung inflammation were significantly reduced with the present invention treating a cotton rat for influenza.

FIG. 2A shows the reduction of IFNγ.

FIG. 2B shows the reduction of GRO/KC.

FIG. 2C shows the reduction of MIP2.

FIG. 2D shows the reduction of TNFα.

FIG. 2E shows the reduction of IL-1β.

FIG. 2F shows the reduction of MCP1.

FIG. 2G shows the assay for TGFβ.

FIG. 2H shows the assay for GMCSF.

FIG. 3A shows that in those CF patients whose TRADD mRNA levels in bronchial brush biopsies are relatively high, the rate of lung function decline is faster.

FIG. 3B shows an enlarged inset of FIG. 3A.

FIG. 4A shows that in those patients whose IL-8 mRNA levels in the same brush biopsies are relatively high, the rate of lung function decline is also faster.

FIG. 4B shows an enlarged inset of FIG. 4A.

FIG. 5 shows the high degree of correlation between mRNA levels of IL-8 and TRADD, across the entire range of FEV1, % Predicted values (R2=0.81).

FIG. 6 is a micrograph of a brush biopsy of human bronchial epithelial cell with cystic fibrosis.

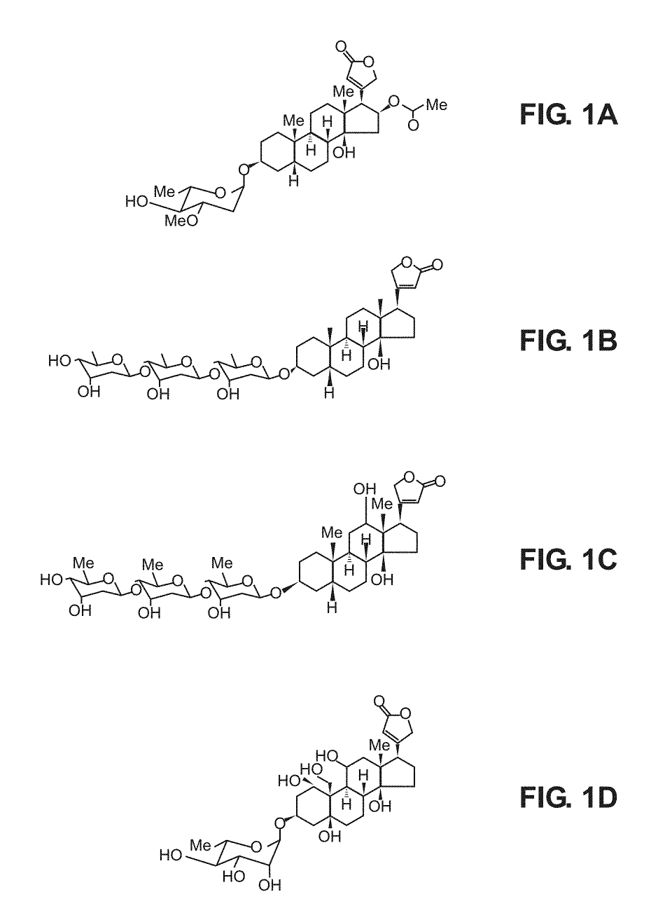

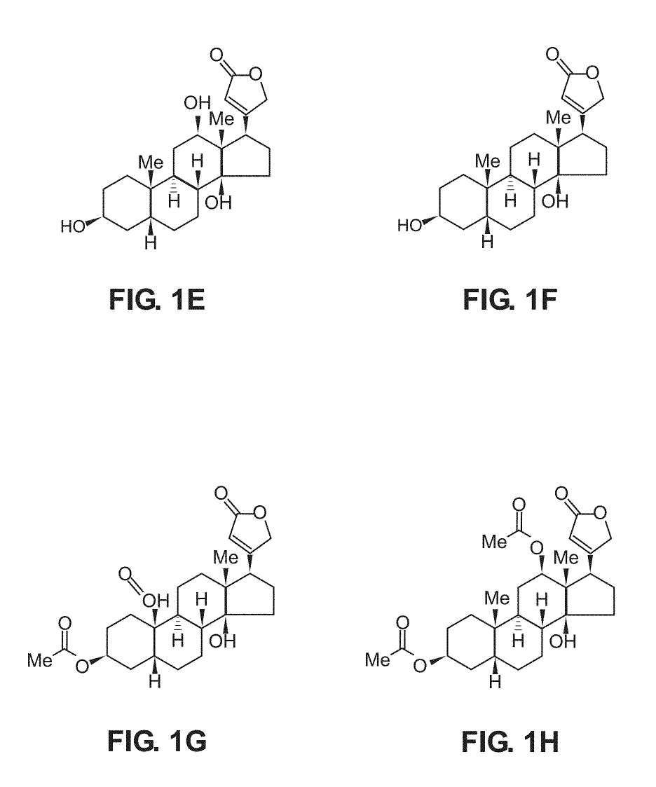

### DETAILED DESCRIPTION OF THE PREFERRED EMBODIMENTS

The following description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications in the preferred embodiments will be readily apparent to one skilled in the art, and the general principals defined herein may be applied to other embodiments and applications without departing from the scope of the invention. The present invention is not intended to be limited to the embodiments shown, but is to be accorded the widest possible scope consistent with the principals and features disclosed herein.

In one embodiment the cardiac glycoside compounds of Table I are administered to a mammal for the treatment of cystic fibrosis, or other forms of acute or chronic obstructive pulmonary disease.

In another embodiment the cardiac glycoside compounds of Table 1 are administered to a mammal for the treatment of cystic fibrosis, or other forms of acute or chronic obstructive pulmonary disease, using one or more of the cardiac glycoside compounds of Table 1.

Another aspect of the invention provides a method for treating cystic fibrosis, other forms of acute or chronic obstructive pulmonary disease, in a mammal by administering to a mammal a therapeutically effective amount of one or more cardiac glycoside compounds typified by those shown in Table 1 of the present invention. Administration of the cardiac glycoside compounds(s) may occur prior to the manifestation of symptoms characteristic of cystic fibrosis, such that the symptoms of cystic fibrosis are prevented, or alternatively, delayed in its progression.

Another aspect of the invention provides a method for being included as an adjuvant to existing anti-cystic fibrosis drugs, or anti-cystic fibrosis drugs which may be developed in the future.

The term “therapeutically effective amount,” as used herein, is that amount that achieves at least partially a desired therapeutic or prophylactic effect in the symptoms of cystic fibrosis, or other forms of acute or chronic obstructive pulmonary disease. The amount of cardiac glycoside compound necessary to bring about prevention and/or therapeutic treatment of cystic fibrosis or related condition, is not fixed per se. An effective amount is necessarily dependent upon the identity and the form of the cardiac glycoside employed, the extent of the protection needed, or the severity of the cystic fibrosis condition.

Cardiac glycoside drugs include, but are not limited to, digitoxin, oleandrin, and digoxin.

In conjunction with the prophylactic or therapeutic treatment, pharmacogenomics (i.e., the study of the relationship between an individual's genotype and the individual's response to a foreign compound or drug) may be considered. Differences in metabolism of therapeutics can lead to severe toxicity or therapeutic failure by altering the relation between dose and blood concentration of the pharmacologically active drug. Thus, a physician or a clinician may consider applying knowledge obtained in relevant pharmacogenomics studies in determining whether to administer a cardiac glycoside compound as well as tailoring the dosage and/or therapeutic regimen of treatment with a cardiac glycoside compound.

Pharmacogenomics deals with clinically significant hereditary variations in the response to drugs due to altered drug disposition and abnormal action in affected persons. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is hemolysis after ingestion of oxidant drugs (anti-malarials, sulfonamides, analgesics, nitro furans) and consumption of fava beans.

One pharmacogenomics approach to identifying genes that predict drug response, known as a “genome-wide association,” relies primarily on a high-resolution map of the human genome consisting of already known gene-related sites (e.g., a “bi-allelic” gene marker map which consists of 60,000-100,000 polymorphic or variable sites on the human genome, each of which has two variants). Such a high-resolution genetic map can be compared to a map of the genome of each of a statistically substantial number of subjects taking part in a Phase II/III drug trial to identify genes associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process. However, the vast majority of SNPs may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals. Thus, mapping of the cardiac glycoside compounds of the invention to SNP maps of patients may allow easier identification of these genes according to the genetic methods described herein.

Alternatively, a method termed the “candidate gene approach,” can be utilized to identify genes that predict drug response. According to this method, if a gene that encodes a drug target is known, all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the established standard and safe dose of a drug. These polymorphisms are expressed in two phenotypes in the population, the poor metabolizer and the extensive metabolizer. The prevalence of a poor metabolizer phenotypes is different among different populations. For example, the gene coding for CYP2D6 is highly polymorphic and several mutations have been identified in poor metabolizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabolizers show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification. Alternatively, a method termed the “gene expression profiling” can be utilized to identify genes that predict drug responses. For example, the gene expression of an animal dosed with a drug (e.g., in response to a cardiac glycoside molecule of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a mammal with a cardiac glycoside compound.

The invention is further directed to pharmaceutical compositions comprising one or more cardiac glycoside molecule(s) of the present invention and a pharmaceutically acceptable carrier.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, hemectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol).

A Clinical Trial has been performed to test the results of using a cardiac glycoside to treat cystic fibrosis. The Clinical Trial described herein using a cardiac glycoside to treat cystic fibrosis found the drug made a statistically significant improvement in the patients, as pulmonary exacerbations were significantly reduced during the treatment. Certainly other levels of inflammatory markers were affected as well, as demonstrated by the reduction in neutrophils.

It is of interest to note that the clinical trial to use digitoxin to treat cystic fibrosis patients has shown some benefit to the treatment. The clinical trial had no serious adverse events. The only adverse events reported were said to be the same events that would be expected from any cystic fibrosis patient (headaches, abdominal pain, nausea, pulmonary exacerbation, but there were no clinically significant cardiac arrhythmias, changes in cardiac status, or 24 hour Holter monitor abnormalities. This may be the first clinical trial treating humans for IL-8 inflammation with digitoxin. Prior human treatments with digitoxin have been for cardiac diseases.

Additionally, there was an effect on Pulmonary, upper respiratory events calculated after the 28 days of daily dosing. The placebo 8 patient's cohort had 11 pulmonary, upper respiratory events. The 8 patients dosed at 0.05 mg/day digitoxin had 9 pulmonary, upper respiratory events. The 8 patients dosed with 0.10 mg/day digitoxin had only 2 events in the 28 days.

The analysis follows:

The reduction in adverse events from the placebo (11) to the low dose of (0.05 mg/day digitoxin) is not statistically significant.

The reduction in adverse events from the placebo (11) to the high dose of 0.10 mg/day digitoxin (2) is statistically significant at p<=0.00129.

The reduction in adverse events from the low dose (9) to the high dose (2) is also statistically significant at p<=0.0386.

The cardiac glycosides can be used alone or as an adjuvant therapy with other drugs that may be supportive in lowering inflammation and improving the function of the CFTR protein and to enhance the general well being of the patient. These drugs include, but are not limited to: C4-Ceramide, and microRNAs 1, 16, 302a, ivacaftor, lumacaftor, ibuprofen, quertecin, the tumeric extract curcumin, the enzyme DNASE, and corticosteroids.

MicroRNA 155 is a well-studied microRNA, that is known to increase levels of IL-8 when the microRNA 155 is hypersecreted. When microRNA 155 is reduced by giving an antagomiR, the levels of IL-8 are also reduced.

### EXAMPLES

Example #1

### Demonstration that Treatment of a Patient with Digitoxin Reduces the Frequency of Pulmonary Exacerbations

Experiment #1: Study design, methods, and basis for patient selection.

Experiment# 1.1. Study design and methods: The study was a randomized, double-blind, placebo controlled, repeat dosing escalation trial. Adults 18-45 years of age with a diagnosis of CF and meet Inclusion/exclusion criteria were selected for the trial. The 24 evaluable patients were studied at a single site, and randomized with respect to drug treatment and placebo at a ratio of 2:1, respectively. Screening included a 24 hour Holter monitor to screen for possible for baseline dysrhythmias. Periodic assessments included a 24 hour Holter cardiac monitor, spirometry, pulse oximetry, ECG, induced sputum, nasal cell collection, vital signs, physical exam, serum concentrations and PK studies of digitoxin, safety labs and pregnancy testing. Dosing was once a day for 28 days with 8 patients in each group. The 3 groups included (i) digitoxin 0.05 mg by mouth, each day, for 28 days, defined as low dose; (ii) digitoxin 0.10 mg by mouth, each day, for 28 days, defined as high dose; and (iii) placebo, administered every day for 28 days.

Experiment #1.2. Inclusion and exclusion criteria:

The inclusion criteria included (i) male or female, 18 to 45 years of age; (ii) confirmed diagnosis of CF by sweat test (a sweat chloride concentration≧60 meq/L), and a genotype consistent with a CF diagnosis; (iii) FEV 1, % PRED≧40%; (iv) clinically stable with no evidence of acute upper respiratory or lower respiratory tract infection or current pulmonary exacerbation; (v) ability to perform spirometry; (vi) weight≧45 kg at screening and at visit #1 (dosing).

The exclusion criteria included: (i) use of an investigational agent within 4 weeks before screen; (ii) use of a medication with an anti-neutrophil or anti-inflammatory or immunosuppressive agent within 4 weeks prior to day 1; (iii) Use of a topical nasal steroid 2 weeks prior to day 1; (iv) unable to stop macrolide antibiotics 4 weeks prior to day 1; (v) history of significant cardiac disease or cardiac arrhythmia; (vi) pulmonary hypertension; (vii) Burkholderia species in sputum within 2 years of screening visit; (viii) unwilling to use a beta agonist (or levalbuterol) prior to induced sputum procedures; (ix) oxygen saturation<92% on room air at the screening visit; (x) pregnant, breast feeding, unwilling to use effective birth control; (xi) significant history of hemoptysis≧60 cc per episode during the 30 days prior to screening visit; (xii) significant history of hepatic, cardiovascular, renal, neurological, hematologic, or peptide ulcer disease or clinically significant lab results; (xiii) presence of a condition or abnormality that in the opinion of the physician would compromise the safety of the subject or the quality of the data.

The demographics of the subjects/patients selected for the trial are shown in Table 2, which depicts patient Demographics and Trial Design.

The average data for the three groups correspond to the inclusion/exclusion criteria, and are quite similar to one another across the board.

TABLE 2
CF Patient Demographics* and Trial Design**

| | Placebo. | Dig (0.05 mg). | Dig (0.10 mg). |
|---|---|---|---|
| Demographic | n = 8 | n = 8 | n = 8 |
| Age | 24.8 | 23.5 | 30.3 |
| | (18.2-39.7) | (18.2-37.7) | (19.8-42.8) |
| Ethnicity (Cauc) | 8 | 7 | 8 |
| Gondor (F) | 4 | 6 | 4 |
| Weight (kg) | 63.6 | 62.6 | 72.2 |
| Height (cm) | 166.5 | 170.8 | 171.4 |
| BMI (Kg/m2) | 22.8 | 21.4 | 25.2 |
| FEV1 % Pred | 82.1 | 68.9 | 79.7 |
| (mean) | | | |
| FEV1. % Pred | 87.0 | 72.0 | 72.5 |
| (median) | | | |

*Homozygous [ΔF508]CFTR;
**Randomized, double blind placebo-controlled trial

Experiment #2: Test of ability of digitoxin to reduce pulmonary/upper respiratory adverse events. Table 3 shows a summary of adverse events observed during the course of daily administration of either placebo, digitoxin (0.05 mg) or digitoxin (0.10 mg), over a 28-day period. During this period, subjects were not taking macrolide antibiotics. The patients getting the placebo had 11 pulmonary/upper respiratory events. There were somewhat fewer Adverse Events (AEs) in other systems. The patients receiving the low dose of digitoxin (0.05 mg digitoxin) had 9 adverse events associated with the pulmonary and upper respiratory system. The patients receiving the high dose of digitoxin (0.10 mg digitoxin) had only 2 adverse events associated with the pulmonary and upper respiratory system.

TABLE 3
Summary of Adverse Events after Daily
Dosing with Digitoxin for 28 days.

| | | Dig | Dig | |
|---|---|---|---|---|
| | Placebo, | (0.05 mg), | (0.10 mg), | Total, |
| SystemAEs | n · 8 | n = 8 | n = 8 | n = 24 |
| Pulm, upper_resp | 11 | 9 | 2 | 22 |
| Neurological | 3 | 8 | 2 | 13 |
| Gastrointestinal | 7 | 5 | 5 | 17 |
| Musculoskel | 0 | 2 | 1 | 3 |
| Renal/GU | 1 | 1 | 1 | 3 |
| Reproductive | 0 | 1 | 0 | 1 |
| Dermatolog | 1 | 0 | 1 | 2 |
| General | 4 | 3 | 3 | 10 |
| Total | 27 | 29 | 15 | 71 |

Experiment #3: Test for statistical significance of drug effects on frequency of pulmonary/upper respiratory adverse events.

Table 4 shows that when comparing low dose digitoxin (0.05 mg) with placebo, there were 9 cases of pulmonary/upper respiratory AEs, compared with 11 for the placebo-treated group. Using a statistical instrument which yields a 2-sided p-value based on exact binomial probability distribution, the 1-tailed t-test yielded a P value of 0.3318, while the 2-tailed t-test yielded a P value of 0.6636. The conclusion is that we are insufficiently powered to discriminate between placebo and low dose 0.05 mg digitoxin treatment for reducing pulmonary/upper respiratory AEs.

TABLE 4
Significance of Treatment with 0.05 mg Digitoxin
Daily for 28 days on Pulmonary Adverse Events

| Adverse | Digitoxin, | | |
|---|---|---|---|
| Events | 0.05 mg | Placebo | Total |
| Cases | 9 | 11 | 20 |
| Person-Time* | 224 | 224 | 448 |
| Incidence | 0.0401786 | 0.0491071 | 0.0446429 |
| Rate | | | |

| | Point | |
|---|---|---|
| | estimate | [95% Conf. Interval] |
| Inc. rate diff. | −0.0089286 | −.048059 → 0.030219 |
| Inc. rate ratio | 0.08181818 | 0.2996767 → 2.171803 (exact) |
| prcv. frac. ex. | 0.1818182 | −1.171803 → 0.7003233 |
| | | (exact} |
| Prev. frac. pop | 0.0909091 | |
| | (midp) Pr (k <= 9)= | P = 0.3318 (exact) |
| | midp) 2 * Pr (k <= 9)= | P = 0.6636 (exact)** |

*Person-time = number of subjects × number of days (viz, 8 × 28 = 224)
**2-sided p-value based on exact binomial probability distribution

Table 5 shows that when comparing high dose digitoxin (0.10 mg) with placebo, there were only 2 cases of pulmonary/upper respiratory AEs, compared with 11 for the placebo-treated group. Using a statistical instrument which yields a 2-sided p-value based on exact binomial probability distribution, the 1-tailed t-test yielded a P value of 0.0065, while the 2-tailed t-test yielded a P value of 0.0129. The conclusion is that we are sufficiently powered to discriminate between placebo and high dose 0.10 mg digitoxin treatment for reducing pulmonary/upper respiratory AEs.

TABLE 5
Significance of treatment with 0.10 mg digitoxin
daily for 28 days on Pulmonary Adverse Events

| Adverse | Digitoxin, | | |
|---|---|---|---|
| Events | 0.10 mg | Placebo | Total |
| Cases | 2 | 11 | 13 |
| Person-Time* | 224 | 224 | 448 |
| Incidence | 0.0089286 | 0.0491071 | 0.0290179 |
| Rate | | | |

| | Point | |
|---|---|---|
| | estimate | [95% Conf. Interval] |
| Inc. rate diff. | −0.0401781. | −0.0717266 →−.0086306 |
| Inc. rate ratio | 0.1818182 | 0.0195828 → 0.8330833 (exact} |
| prev. frac. ex. | 0.8181818 | 0.1669167 → 0.9804172 (exact} |
| Prev. frac: pop | 0.4090909 | |
| | (midp) Pr (k <= 9)= | P = 0.0065 (exact) |
| | midp) 2 * Pr (k <= 9)= | P = 0.0129 (exact)** |

*Person-time = number of subjects × number of days (viz, 8 × 28 = 224)
**2-sided p-value based on exact binomial probability distribution

Table 6 shows that when comparing high dose digitoxin (0.10 mg) with low dose digitoxin (0.05 mg), there were 2 cases of pulmonary/upper respiratory AEs for the high dose, compared to 9 instances of pulmonary/upper respiratory AEs for the low dose digitoxin (0.05 mg). Using a statistical instrument which yields a 2-sided p-value based on exact binomial probability distribution, the 1-tailed t-test yielded a P value of 0.0193, while the 2-tailed t-test yielded a P value of 0.0386. The conclusion is that we are sufficiently powered to discriminate between low dose 0.05 mg digitoxin and high dose 0.10 mg digitoxin treatment for reducing pulmonary/upper respiratory AEs.

TABLE 6
Significance of treatment with 0.10 mg digitoxin
vs 0.05 mg digitoxin daily for 28 days on Pulmonary

| Adverse Events | | | |
|---|---|---|---|
| Adverse | Digitoxin, | Digitoxin, | |
| Events | 0.10 mg | 0.05 mg | Total |
| Cases | 2 | 9 | 11 |
| Person-Time• | 224 | 224 | 448 |
| Incidence | 0.0089286 | 0.0401786 | 0.0245536 |
| Rate | | | |

| | Point | |
|---|---|---|
| | estimate | (95% Conf. Interval) |
| Inc. rate diff. | −0.03125 | 0.0602699 → .0022301 |
| Inc. rate ratio | 0.2222222 | 0.0233646 → 1.073638 |
| | | (exact) |
| prev. frac. ex. | 0.7777778 | 0.0736384 → 0.9766354 |
| | | (exact) |
| Prev. frac. pop | 0.3888889 | |
| | (midp) Pr (k <= 9)= | P = 0.0193 (exact) |
| | midp) 2 * Pr (k <= 9J= | P = 0.0386 (exact)** |

*Person-time = number of subjects × number of days (viz, 8 × 28 = 224)
**2-sided p-value based on exact binomial probability distribution

Experiment #4: Test for statistical significance of drug effects on frequency of all adverse events.

Table 7 shows that when comparing high dose digitoxin (0.10 mg) with placebo for all Adverse Events, there were 15 cases of AEs, compared with 27 for the placebo-treated group. Using a statistical instrument which yields a 2-sided p-value based on exact binomial probability distribution, the 1-tailed t-test yielded a P value of 0.0330, while the 2-tailed t-test yielded a P value of 0.0660. The conclusion is that we are very close to being sufficiently powered to discriminate between placebo and high dose 0.10 mg digitoxin treatment for reducing the frequency all AEs.

TABLE 7
Significance of Treatment with 0.10 mg
Digitoxin Daily for 28 days on All Adverse Events

| Adverse | Digitoxin, | | |
|---|---|---|---|
| Events | 0.10 mg | Placebo | Total |
| Cases | 15 | 27 | 42 |
| Person-Time• | 224 | 224 | 448 |
| Incidence | 000.066964-3 | 0.1205357 | 0.09375 |
| Rate | | | |

| | Point | |
|---|---|---|
| | estimate | [95% Cont. Interval) |
| Inc. rate diff. | −0.0535714 | .0.1102769 → −.003134 |
| Inc. rate ratio | 0.5555556 | 0.2747098→ 1.082203 |
| | | (exact) |
| prev. frac. ex. | 0.4444444 | 0.0822031→ 0.7252902 |
| | | (exact) |
| Prev. frac. pup | 0.2222222 | |
| | (midp) Pr (k <= 9)= | P = 0.0330 (exact) |
| | midpl z * Pr (k <= 9)= | P = 0.0660 (exact)** |

*Person-time = number of subjects × number of days (viz, 8 × 28 = 224)
**2-sided p-value based on exact binomial probability distribution

Table 8 shows that when comparing low dose digitoxin (0.05 mg) with placebo for all adverse events, there were 29 cases of pulmonary/upper respiratory AEs on low dose digitoxin, compared with 27 for the placebo-treated group. Using a statistical instrument which yields a 2-sided p-value based on exact binomial probability distribution, the 1-tailed t-test yielded a P value of 0.03957, while the 2-tailed t-test yielded a P value of 0.7914. The conclusion is that we are insufficiently powered to discriminate between placebo and low dose 0.05 mg digitoxin treatment for reducing both pulmonary/upper respiratory AEs (see Table 4), and all AEs (see Table 8).

TABLE 8
Significance of Treatment with 0.05 mg Digitoxin
Daily for 28 days on All Adverse Events

| Adverse | Digitoxin, | | |
|---|---|---|---|
| Events | 0.05 mg | Placebo | Total |
| Cases | 29 | 27 | 56 |
| Person-Time* | 224 | 224 | 448 |
| Incidence | 0.1294643 | 0.1205357 | 0.125 |
| Rate | | | |

| | Point | |
|---|---|---|
| | estimate | (95% Conf. Interval) |
| Inc. rate diff. | −0.0089286 | −0.0565492 → −.0744064 |
| Inc. rate ratio | 1.074074 | 0.6137252 → 1.885546 |
| | | (exact} |
| prev. frac. ex. | 0.0689655 | 0.6293938 → 0.4696496 |
| | | (exact) |
| Prev. frac. pop | 0.0357143 | |
| | (midp) Pr (k <= 9)= | P = 0.3957 (exact) |
| | midp) Pr (k <= 9J= | P = 0.7914 (exact)** |

Person-time = number of subjects × number of days (viz, 8 × 28 = 224)
**2-sided p-value based on exact binomial probability distribution

Table 9 shows that when comparing high dose digitoxin (0.10 mg) with low dose digitoxin (0.05 mg) for all AEs, there were 15 cases of all AEs for the high dose, compared to 29 instances of all AEs for the low dose digitoxin (0.05 mg). Using a statistical instrument which yields a 2-sided p-value based on exact binomial probability distribution, the 1-tailed t-test yielded a P value of 0.0178, while the 2-tailed t-test yielded a P value of 0.0357. The conclusion is that we are sufficiently powered to discriminate between low dose 0.05 mg digitoxin and high dose 0.10 mg digitoxin treatment for reducing all AEs.

TABLE 9
Significance of Treatment with 0.10 mg vs 0.05 mg
Digitoxin Daily for 28 days on All Adverse Events

| Adverse | Digitoxin, | Digitoxin, | |
|---|---|---|---|
| Events | 0.10 mg | 0.05 mg | Total |
| Cases | 15 | 29 | 44 |
| Person-Time* | 224 | 224 | 448 |
| Incidence | 0.0669643 | 0.1294643 | 0.0982143 |
| Rate | | | |

| | Point | |
|---|---|---|
| | estimate | [95% Conf. Interval] |
| Inc. rate diff. | −0.0625 | −0.1205399 → −.0044601 |
| Inc. rate ratio | 0.5172414 | 0.2577309 → 0.9967851 (exact) |
| prev. rrac. ex. | 0.4827586 | 0.0032149 → 0.7422691 (exact) |
| Prev. frac. POP | 0.2413793 | |
| | (midp) Pr (k <= 9)= | P = 0.0178 (exact) |
| | midp) 2 * Pr (k <= 9)= | P = 0.0357 (exact)•• |

*Person-time = number of subjects × number of days (viz, 8 × 28 = 224)
**2-sided p-value based on exact binomial probability distribution

Chronic Obstructive Pulmonary Disease (COPD) is an environmentally induced form of Cystic Fibrosis, caused by components of cigarette smoke.

The preponderance of evidence suggests that the symptoms of COPD are due to acquired loss of CFTR function, and that lessons learned from CF may be applicable to COPD, as well as other airway diseases [62 Dransfield M T, 63 Mall M A]. The parallels with CF also persist even to the extent of efficacy of the CF potentiator drug ivacaftor (VX-770). This drug partially reverses the functional CFTR loss in pulmonary epithelia caused by smoking [64 Sloane P A]. Extra-pulmonary effects of COPD-dependent-CFTR loss have also been observed. For example, reduced beta adrenergic dependent sweat chloride, and increased sweat chloride concentration, both phenotypical of CF, have also recently been reported in smokers [65]. Furthermore, these investigators show that the effect persists in spite of smoking cessation [65 Courville C A]. Intestinal chloride currents are also significantly reduced in smokers, as well as in cigarette smoke-exposed mice [66 Raju S V]. Thus, wherever CFTR is expressed, COPD patient experience a reduction in CFTR expression which has symptomatic consequences.

There are many reports that cancer can be promoted by the misfolded gene CFTR. Thus, a side benefit of this treatment is that cancer promotion can be stopped by correcting the misfolded, dysregulated gene of CFTR.

Inhibition of Pro-Inflammatory Markers by Digitoxin in Rodent Influenza:

Applicant previously conducted a study on a mammal, a rodent with induced lung inflammation from Wuhan influenza virus. It was found the treated animals had the consequence of a significant reduction in cytokines and chemokines from the digitoxin treatment (data from unpublished result from 2006 follows).

Experiment and Results: Inflammation was induced in the lung of a non-CF laboratory rodent to further investigate the action of digitoxin.

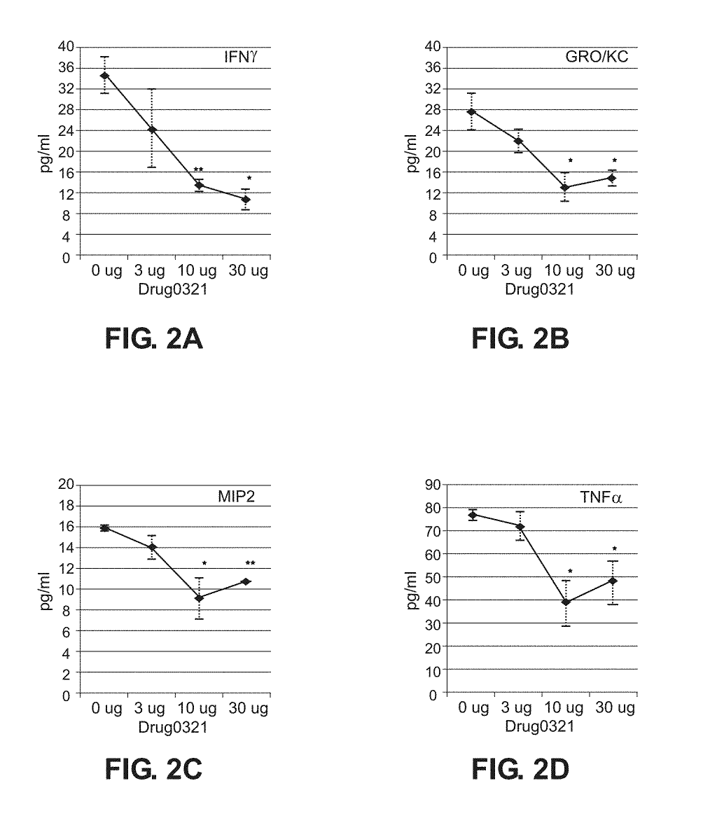

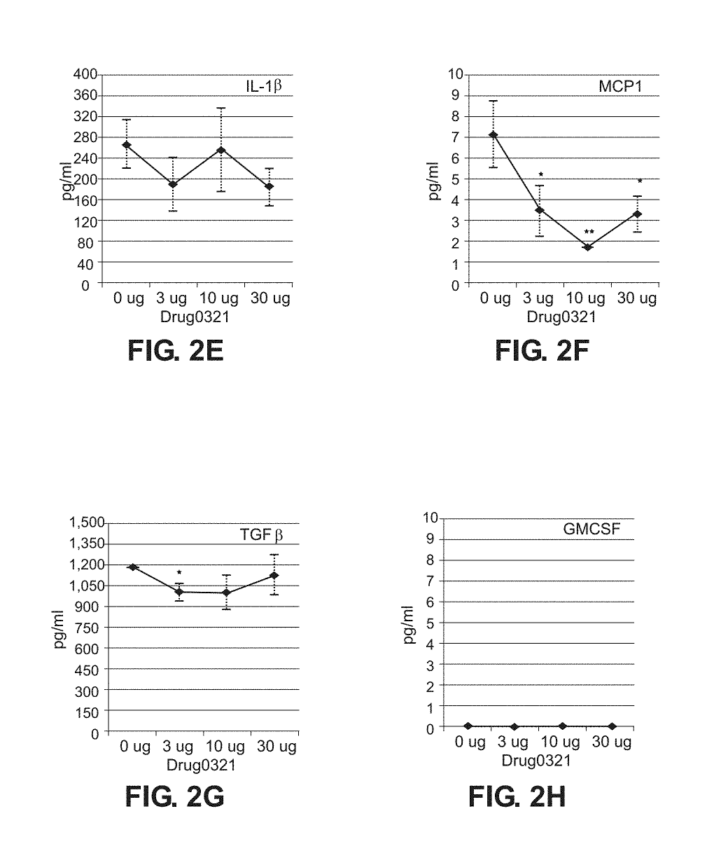

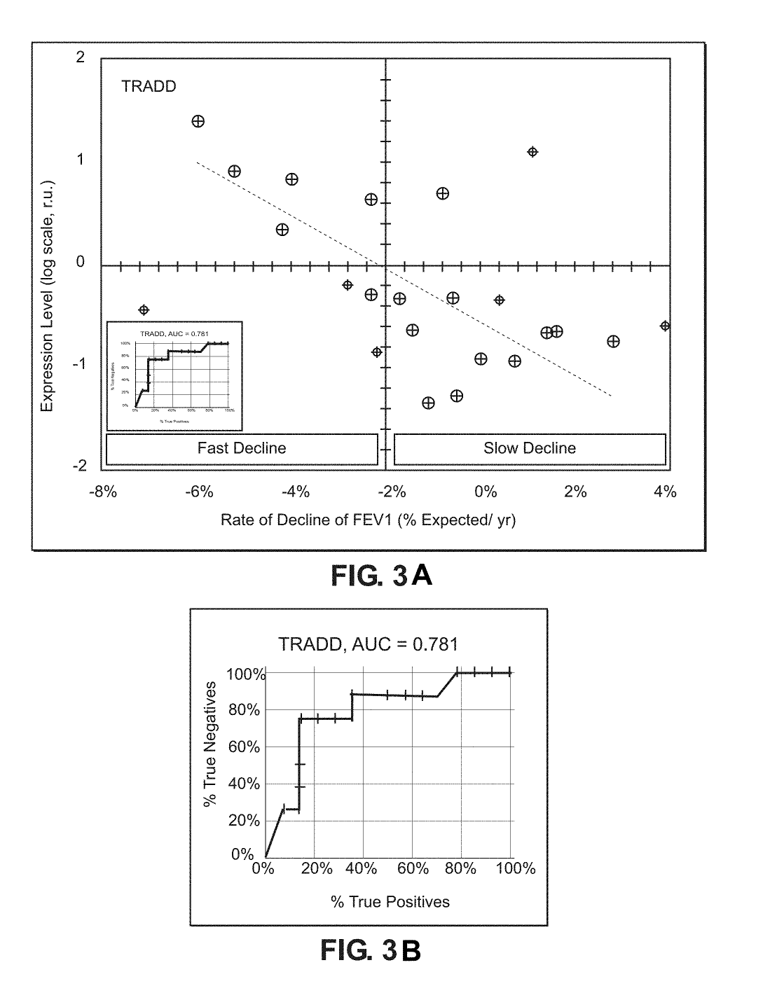

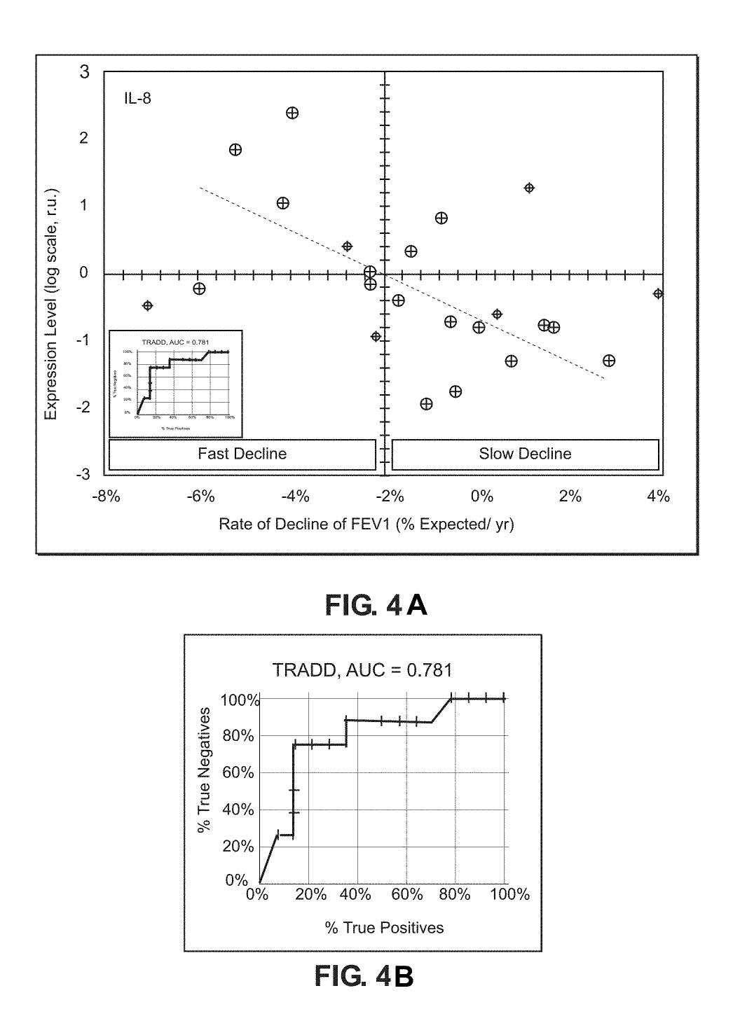

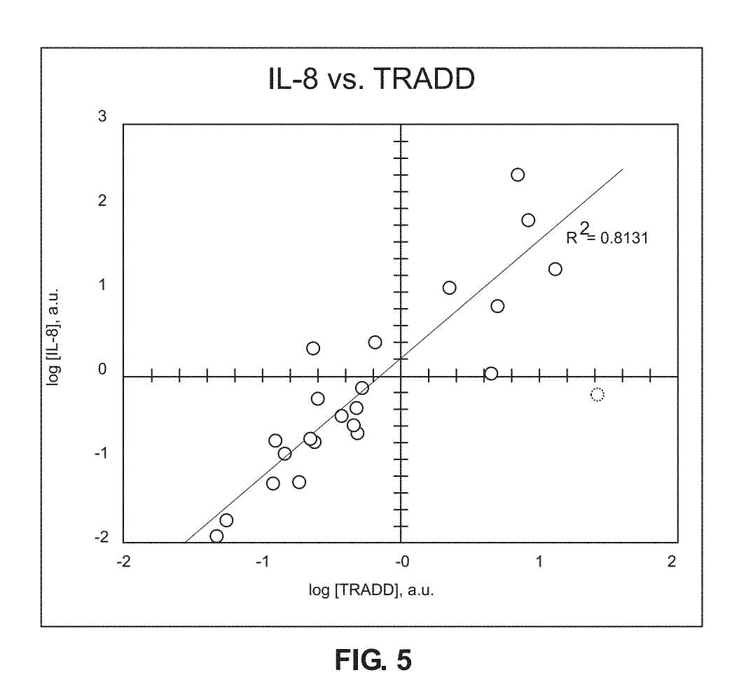

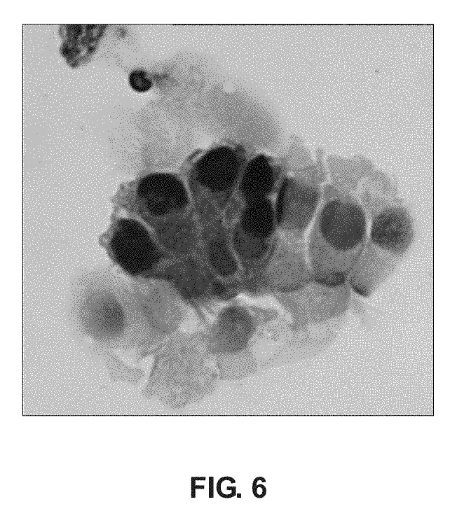

Experiment and Results: Inflammation was induced in the lung of a non-CF laboratory rodent to further investigate the action of digitoxin on lung inflammation. The rodent model was pre-treated on the preceding day with 3, 10 and 30 mg/kg of digitoxin, and treatment sustained on a daily basis for 7 days. Lung tissue was harvested on day 7, and assayed in terms of pg/ml for iFN-Gamma, GRO/KC, MTP-2, TNF-ALPHA, IL-1B, MCP-1. TGF-Beta, and GM-CSF. Rodents lack IL-8, and instead have GRO/KC and MIP-2. Data in FIG. 2 show significant reductions were observed for GRO/KC, MTP-2, IFN-gamma, TNF-alpha, and MCP-1. No significant changes were seen for IL-1B and TGF-B. We were unable to detect GM-CSF in the tissue under any of the conditions.

Interpretation: These data show that digitoxin can induce a profound, dose-dependent reduction in an experimental model of lung inflammation in a rodent model. As anticipated, GRO/KC and MIP-2, the rodent equivalent of IL-8, were suppressed by digitoxin. Certain other proinflammatory analytes were also reduced, including TNF-alpha. Therefore, this alternative model represents a second, positive test of the anti-inflammatory power of low doses of digitoxin in vivo.

Methods: The assays for different cytokines and chemokines were performed with commercial micro-sandwich ELISA assays. (Searchlight System, Pierce-Thermo). The results shown are for 22 patients, including 14 females and 8 males.

What is claimed is:

1. A small molecule inhibitor for reducing raised cytokines and chemokines caused by the protein product of the cystic fibrosis mutant gene, wherein the inhibitor is selected from the group consisting of:
a) a cardiac glycoside compound such as but not limited to digitoxin, digoxin, ouabain, oleandrin, digoxigenin, acetyldigitoxins, acetyldigoxins, cymarine, digitoxigenin, digoxigenin, medigoxin, neoconvalloside, ouabain, strophanthins, strophanthidin and acetyl-strophantidin, and other related compounds such as marinobufagenin, used either alone or in combination with:
b) a microRNA such as microRNA 1, microRNA 16, or microRNA 302a optionally combined with:
c) a yeast extract such as C4-Ceramide.

2. The use of the inhibitor of claim 1 for treating a disorder caused by a mutant CFTR gene expressing a mutant protein such as but not limited to [ΔF508], in the disease of cystic fibrosis (CF).

3. The use of the inhibitor of claim 1 for treating a disorder caused by an environmentally induced mutant CFTR gene, such as that induced by smoking, which causes the expression of a mutant CFTR protein in the lung and concomitant high levels of inflammatory markers such as cytokines and chemokines like IL-8, in the disorder chronic obstructive pulmonary disorder (COPD).

4. The use of the inhibitor of claim 1 to reduce IL-8 and other immune system indicators of inflammation such as, but not limited to, reduction of neutrophils, and concomitantly reducing pulmonary exacerbations and other adverse events associated with cystic fibrosis (CF).

5. The use of the inhibitor of claim 4 to reduce TNF, leading to fewer neutrophils, and other immune system cells, and reducing neutrophil elastase and selectin ligand (SLIG), an adhesion molecule, and other immune system components of inflammation such as integrin (INT), and concomitantly reducing pulmonary exacerbations and other adverse events associated with cystic fibrosis (CF).

6. The use of the inhibitor of claim 4 to reduce TNF, leading to fewer neutrophils, and other immune system cells, and reducing neutrophil elastase and selectin ligand (SLIG), an adhesion molecule, and other immune system indicators of inflammation such as integrin (INT), and concomitantly reducing pulmonary exacerbations and other adverse events which results in treating pulmonary exacerbations in chronic obstructive pulmonary disease (COPD) and asthma.

7. The use of the inhibitor of claim 1 to treat a human individual or other mammal having a disorder comprising a chronic inflammatory system response component involving IL-8 or other chemokine/cytokine or related condition such as raised levels of neutrophils, resulting in a cytokine storm in the individual or mammal, comprising the step of administering to said individual or mammal a therapeutically effective amount of the inhibitor able to reduce levels of IL-8 or other inflammatory markers wherein the inhibition disrupts the inflammation and disrupts or reduces the severity of the disorder.

8. The use of the inhibitor as claimed in claim 7, wherein the inhibitor is digitoxin.

9. The use of the inhibitor as claimed in claim 7, wherein the inhibitor is digitoxin and the therapeutically effective amount of digitoxin is an amount to establish a concentration of the inhibitor of about 0.05 nanoMolar to less than about <5.0 nanoMolar in body fluids in the individual or the mammal.

10. The use of the inhibitor as claimed in claim 7, wherein the therapeutically effective amount for treating pulmonary exacerbations and other CF caused adverse events is 0.05 mg/day to 0.10 mg/day for a person weighing more than about 40 kilograms.

11. The use of the inhibitor as claimed in claim 7, wherein the therapeutically effective amount for treating pulmonary exacerbations and other CF caused adverse events is <0.05 mg/day to <0.10 mg/day for a person weighing less than about 40 kilograms.

12. The use of the inhibitor of claim 1 for treating pulmonary exacerbations in a mammal having cystic fibrosis or a related condition to obtain a reduction in said pulmonary exacerbations associated with a deteriorating lung, such as, but not limited to, reductions in infection, airway obstruction associated with thickened secretions and cellular debris, bronchial hyperactivity, increased cough, increased sputum production, shortness of breath, chest pain, loss of appetite, loss of weight, lung function decline, hemoptysis and/or pneumothorax.

13. The use of the inhibitor of claim 1 for treating pulmonary exacerbations in chronic obstructive pulmonary disease such that there is a reduction in said pulmonary exacerbations associated with a deteriorating lung, such as, but not limited to, reductions in infection, airway obstruction associated with thickened secretions and cellular debris, bronchial hyperactivity, increased cough, increased sputum production, shortness of breath, chest pain, loss of appetite, loss of weight, lung function decline, hemoptysis and/or pneumothorax.

14. A pharmaceutical composition for treating pulmonary exacerbations in chronic obstructive pulmonary disease or a related condition, wherein the composition comprises a cardiac glycoside compound and a pharmaceutically acceptable carrier.

15. The use of the compound of claim 14 to treat pulmonary exacerbations in chronic obstructive pulmonary disease or related condition in a mammal, comprising administering to said mammal a therapeutically effective amount of the pharmaceutical composition.

16. The compound of claim 14, including an inert substance that is compatible with nature.

17. An inhibitor of pulmonary exacerbations, said inhibitor being selected from the family of cardiac glycosides according to claim 1, where the inhibitor is complementary to or has at least 85% sequence identity to a fragment of a molecule from that family which has either been extracted, made with medicinal chemistry, synthetic organic chemistry, or made by chemical engineering or by recombinant techniques.

18. The inhibitor according to claim 1, wherein the inhibitor corresponds to or is complementary to at least an 85% fragment of the microRNA, or has been made from medicinal chemistry, synthetic organic chemistry, or chemical engineering, or by a recombinant technique.

19. The inhibitor of claim 1, wherein the small molecule inhibitor has a modified backbone, substituted sugar moiety, or cholesterol conjugation.

20. The inhibitor of claim 1, wherein the inhibitor reduces the effects of a disorder such as influenza in a mammal or human, not limited to Wuhan strain of influenza, the inhibitor causing the lung to reduce the levels of cytokines and chemokines such as GRO/KC, MIP-2, IFN-gamma, TNF-alpha, and MCP-1.

21. The inhibitor of claim 1, wherein the inhibitor is administered orally, in the eyes, in the nose, intravascularly, intramuscularly, subcutaneously, intraperitoneally, or transdermal.
